(19) United States
(12) Patent Application Publication
(10) Pub. No.: US 2008/0275265 A1
(43) Pub. Date: Nov. 6, 2008

(54) Process for the Preparation of (Aminoalkylamino)Alkyl Halides and Conversion to Amifostine

(75) Inventor: Edward G. Samsel, Baton Rouge, LA (US)

Correspondence Address: ALBEMARLE CORPORATION;PATENT DEPARTMENT, 451 FLORIDA STREET, BATON ROUGE, LA, 70801, US

(73) Assignee: ALBEMARLE CORPORATION, Baton Rouge, LA (US)

(21) Appl. No.: 12/092,516

(22) PCT Filed: Nov. 1, 2006
(86) PCT No.: PCT/US2006/042761

§ 371 Date: May 2, 2008

Related U.S. Application Data
(60) Provisional application No. 60/734,007, filed on Nov. 3, 2005.

(51) Int. Cl. C07F 9/165; C07C 209/74

(52) U.S. Cl. 558/146; 564/496; 558/87

(57) ABSTRACT

The present invention relates to processes for the preparation of (ω-aminoalkylamino)alkyl halides, their conversion to S-ω-(ω-aminoalkylamino)alkyl phosphothioates, and purification of the crystalline products of the reaction. The preparation process for the (ω-aminoalkylamino)alkyl halides comprises contacting an appropriate alcohol with a brominating agent in the presence of a sulfone solvent under temperature and pressure conditions suitable to effect salt formation without subsequent premature precipitation. The process is especially useful for converting (ω-aminoalkylamino)ethyl alcohol to amifostine.

### FIELD OF THE INVENTION

The present invention provides processes for the preparation of (ω-aminoalkylamino)alkyl halides, particularly 2-(3-aminopropylamino)ethyl bromide dihydrobromide and its subsequent conversion to and purification of S-ω-(ω-aminoalkylamino)alkyl dihydrogen phosphorothioates, such as amifostine monohydrate and amifostine trihydrate.

### DESCRIPTION OF RELATED ART

As the incidence of cancer and related disorders necessitating chemotherapy and/or radiotherapy increase, interest in radioprotectors which reduce the biological effects of ionizing radiation, including lethality, mutagenicity, and carcinogenicity has grown. One of the most heavily studied groups of radioprotectors, the aminothiols, has been used clinically to minimize damage to normal tissues in cancer chemotherapy. One of the most studied of these compounds, WR-2721 (S-2-(3-aminopropylamino)ethyl dihydrogen phosphorothioates), also called amifostine, Ethyol®, and ethiofos [Grdina, D. J., et al., Cancer Res., 51: pp. 4125-4130 (1991); Kurbacher, C. M.; Mallmann, P. K., Anticancer Research, 18: pp. 2203-2210 (1998)] is now finding use as a protective agent in cancer chemotherapy due to its protective effects with genotoxic chemicals. WR-2721 treatment also offers the prospect of reducing the risk of secondary tumors induced by radiation and chemotherapy.

As a result of the increased interest and need for (ω-aminoalkylamino)alkyl dihydrogen phosphorothioates, such as amifostine, the need for an economic and rapid synthesis method has increased. Generally, the synthesis starts with an (ω-aminoalkylamino)alkyl alcohol being halogenated to produce an (ω-aminoalkylamino)alkyl halide dihydrohalide intermediate, which is then converted to the phosphorothioate final product. While the (ω-aminoalkylamino)alkyl alcohols are commercially available or easily prepared from the corresponding α,ω-alkanediamines, the intermediate (ω-aminoalkylamino)alkyl bromide dihydrobromide salts have proved to be troublesome to prepare in a consistent, economic, and safe manner.

There is substantial literature in the art with respect to processes for the preparation of (ω-aminoalkylamino)alkyl halides. For example, Cortese [Organic Syntheses, Coll. Vol. II; Blatt, A. H., ed.; John Wiley & Sons, Inc., New York, N.Y.; 1943: pp. 91-93], which is herein incorporated by reference, describes the preparation of such compounds using HBr in acetic acid as the brominating agent with heating for an extended period of time. S. Akerfeldt provides a similar approach, with comparable yields [Acta Chem. Scand., 14: pp. 1980-1984 (1960)].

U.S. Pat. No. 3,892,824 to Piper, et al., which is herein incorporated by reference, describes processes for the preparation of antiradiation agents from (ω-aminoalkylamino)alkyl halides, wherein the process recites the reaction of 2-(3-aminopropylamino)ethanol with boiling, 48% hydrobromic acid for an extended period of time (up to two weeks) in order to obtain 80% conversion. These compounds have also been described by the hydrogen bromide cleavage of 3-substituted 2-oxazolidinones [Piper, J. R., et al., Chem. Ind. (London), p. 2010 (1966). A similar process is described by Laduranty, et al. [Bull. Soc. Chim. Belg., 93 (10): pp. 903-912 (1984), which is incorporated by reference herein, wherein the alkyl halide is obtained by treating a phthalimido intermediate with refluxing HBr in acetic acid for 18 hours, with a reported recovery of about 95%.

The problem with using many of these above-described processes in the commercial scale production of (ω-aminoalkylamino)alkyl halides is that these processes take a considerable length of time and often do not have desirable yields. Thus, there exists a need for a process that obtains (ω-aminoalkylamino)alkyl halides in a more efficient manner and in high yield and purity.

### SUMMARY OF THE INVENTION

This invention relates to improved processes for producing (ω-aminoalkylamino)alkyl halides, such as (ω-aminoalkylamino)alkyl bromide dihydrobromides, utilizing a halogenating agent in a sulfone solvent at elevated temperature.

In addition to the process for preparing (ω-aminoalkylamino)alkyl halides from (ω-aminoalkylamino)alkyl alcohols, a process for converting the (ω-aminoalkylamino)alkyl halides into S-ω-(ω-aminoalkylamino)alkyl dihydrogen phosphorothioates, such as amifostine monohydrate and amifostine trihydrate, is also disclosed. Also, this invention relates to a process for preparing purified amifostine monohydrate or amifostine trihydrate from crude amifostine. The process includes the steps of passing an aqueous solution of crude amifostine through at least one activated carbon column, and at least one anion exchange column, adding the purified amifostine solution slowly to a methanol-water solution over a period of time, precipitating amifostine monohydrate or amifostine trihydrate, and isolating the crystalline product.

### DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1 is a ¹H-NMR spectra of 2-(3-aminopropylamino)ethyl bromide dihydrobromide, prepared according to the process of the present invention.

FIG. 2 is a process flow scheme of the purification process for use in the conversion of crude amifostine trihydrate to amifostine monohydrate or trihydrate as described herein.

FIG. 3 is a HPLC chart obtained by the USP monograph method for the crude amifostine monohydrate of Example 4.

FIG. 4 is a HPLC chart obtained by the USP monograph method for the purified amifostine trihydrate of Example 4.

FIG. 5 is a HPLC chart obtained by the USP monograph method for the purified amifostine monohydrate of Example 4.

### DETAILED DESCRIPTION OF THE INVENTION

The present invention addresses the need for alternative methods for commercial scale preparations of (ω-aminoalkylamino)alkyl halides and S-ω-(ω-aminoalkylamino)alkyl dihydrogen phosphorothioates. The methods described herein provide means whereby (ω-aminoalkylamino)alkyl alcohols can be converted to the halides in an efficient manner using a sulfone solvent, which allows the intermediate (dihydrohalide) salt to remain substantially in solution and thereby preventing premature precipitation. By keeping the intermediate in solution, conversion of the intermediate to the desired alkyl halide salt is maximized. Once formed, the alkyl halide salt may be isolated by conventional processes, for example, by precipitation in acetone.

The process for preparing (ω-aminoalkylamino)alkyl halides comprises the steps of:

contacting, in a sulfone solvent, an (ω-aminoalkylamino)alkyl alcohol of Formula (I),

RNH(CH₂)ₘNH(CH₂)ₙ—OH (I),

wherein:
R is hydrogen or a substituted or unsubstituted linear, cyclic, or branched alkyl group having 1 to 12 carbon atoms,
m is an integer from 2 to 8, and
n is an integer from 2 to 6,
   with a first halogenating agent, preferably a brominating agent for a period of time sufficient to provide a dihydrohalide salt of Formula (II)

RNH(CH₂)ₘNH(CH₂)ₙ—OH.2HX (II)

wherein X is a halogen atom, preferably bromine;
contacting, in a sulfone solvent, the dihydrohalide salt of Formula (II) with a second halogenating agent, preferably a brominating agent, for a period of time sufficient to provide an (ω-aminoalkylamino)alkyl halide dihydrohalide salt of Formula (III);

RNH(CH₂)ₘNH(CH₂)ₙ—X.2HX (III); and

subsequently isolating the (ω-aminoalkylamino)alkyl halide dihydrohalide salt of Formula (III).

The process of preparing S-ω-(ω-aminoalkylamino)alkyl dihydrogen phosphorothioates, such as amifostine, comprises the steps of:

contacting the preferred (ω-aminoalkylamino)alkyl bromide dihydrobromide salt of Formula (III) with sodium thiophosphate for a period of time sufficient to form compounds of Formula (IV),

RNH(CH₂)ₘNH(CH₂)ₙSY (IV),

and hydrates thereof, wherein R, m and n are as previously described and Y is PO₃H₂, PO₃HM, or PO₃M₂, with M being an alkali metal selected from sodium, potassium, and lithium.

The crude amifostine prepared by the process described above will contain color bodies and residual sodium thiophosphate upon crystallization. The process for the purifying the crude material to yield an amifostine final product (either monohydrate or trihydrate) generally comprises the steps of preparing an aqueous amifostine solution from crude amifostine and water; contacting the aqueous amifostine solution with at least one anion exchange column and at least one activated carbon column; contacting the purified amifostine solution with a water-alcohol mixture continuously over a period of time from about 0.5 hours to about 9 hours to yield a purified precipitate, wherein the water-alcohol mixture comprises at least about a 1% to about a 60% volumetric excess of alcohol relative to the water; and subsequently isolating the purified amifostine.

### Process

A. Synthesis of (ω-aminoalkylamino)alkyl halides

(ω-aminoalkylamino)alkyl halide compounds of general Formula (III) are prepared in accordance with the following Scheme I:

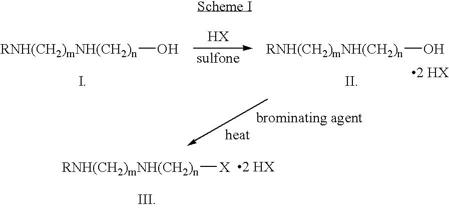

According to this route, an (ω-aminoalkylamino)alkyl alcohol of general Formula (I) is contacted with an acid halide, in a sulfone solvent to produce the alcohol dihydrohalide of Formula (II). This contacting occurs at a temperature between about 100° C. to about 150° C. and a pressure ranging from about 0.5 atm to about 1.5 atm.

The sulfone solvent serves the purpose of allowing the alcohol dihydrohalide of Formula (II) to remain in solution and not prematurely precipitate, a problem typically plaguing previously described methods and associated with low reaction yields. Should the alcohol dihydrohalide precipitate, its conversion to the halide dihydrohalide salt of Formula (III) is attenuate. By keeping the alcohol dihydrohalide in solution, conversion to the halide dihydrohalide salt of Formula (III) is maximized and process can be run more efficiently at elevated temperatures. The molar ratio of sulfone solvent to (ω-aminoalkylamino)alkyl alcohol can range from about 1:1 to about 20:1, and preferably between about 5:1 to about 15:1.

Following the formation of the alcohol dihydrohalide of Formula (II), the alcohol dihydrohalide is contacted with a second halogenating agent, say in the range of from about 100° C. to about 150° C., and a pressure ranging from about 0.5 atm to about 1.5 atm for a period of time sufficient to convert substantially all of the salt of Formula (II) to the halide dihydrohalide salt of Formula (III). The halide salt of Formula (III) can then be isolated by conventional means known in the art, e.g., crystallization. Preferably, the halide dihydrohalide salt/sulfone mixture is combined into a volume of acetone wherein the halide salt precipitates. The precipitate is subsequently filtered, rinsed with additional acetone, and dried with nitrogen.

An example of a typical compound suitable for use as the starting alcohol (Formula I) includes, but is not limited to, 2-(3-aminopropylamino)ethyl alcohol. Such alcohols can be readily obtained from commercial sources, or prepared according to a known procedure, e.g., from the corresponding α,ω-alkanediamines and ethylene oxide by an adaptation of the procedure of Streck, et al. [J. Am. Chem. Soc., 79: pp. 4414 (1957)], which is herein incorporated by reference.

An example of a particular product that can be prepared according to the present invention is 2-(3-aminopropylamino)ethyl bromide dihyrobromide.

Suitable sulfone solvents that may be employed in the processes of the present invention include sulfolane, 2,4-dimethylsulfolane, diphenylsulfolane, and the like. Alternatively, other solvents including N,N-dimethylformamide (DMF), 1-methyl-2-pyrrolidinone (NMP), N,N-dimethylacetamide (DMAC), or mixtures thereof (including mixtures with one or more sulfones) may be used; however, sulfone solvents are preferred. While water can be present in the solvent, or even used as a co-solvent, it is preferred that the system be maintained with less than 0.5% by weight water. Water, when present in the system, tends to increase the amount of by-products formed during the halogenation process. Also, conversion and selectivity are also sacrificed when water is present in the solvent.

The halogenating/brominating agent using in the conversion of (ω-aminoalkylamino)alkyl alcohol of Formula (I) to the halide salt of Formula (II) is typically an acidic halogenating agent. Examples of suitable halogenating/brominating agents for this transformation include but is not limited to hydrogen bromide (HBr) and hydrogen chloride (HCl).

In converting the halide salt of Formula (II) to the halide dihydrohalide salt of Formula (III), any number of halogenating/brominating agents known in the art can be used to effect the transformation, provided that they are stable and do not significantly decompose in the reaction medium. Examples of brominating agents suitable for such use include, but are not limited to, phosphorus tribromide (PBr₃), phosphorus pentabromide (PBr₅), bromoform (CHBr₃), carbontetrabromide (CBr₄), thionyl bromide (SOBr₂), bromine (Br₂) with a phosphine or amine, sodium monobromoisocyanate (SMBI), hydrogen bromide (HBr), and polymeric brominating agents, as well as combinations of V₂O₅ and aq. H₂O₂ under dilute acidic conditions in the presence of alkali bromide salts, as described by Rottenberg, et al. [Org. Proc. Res. Dev., 4 (4): pp. 270-274 (2000)], which is herein incorporated by reference. Preferred brominating agents used in converting the bromides of Formula (II) to the bromide dihydrobromide salts of Formula (III) is phosphorus tribromide (PBr₃) or phosphorus pentabromide (PBr₅). Alternatively, the corresponding chloriding agents may be employed as halogenating agents.

The reaction processes shown in Scheme I may be carried out at temperatures in the range from say about 30° C. to the boiling point of the solvent used. For example, such temperature can range from about 30° C. to about 350° C., preferably between about 100° C. to about 150° C. The reaction processes shown and described in Scheme I can be carried out for a period of time ranging from about 0.1 hour to about 48 hours, however, preferred reaction periods range from about 0.1 hour to about 8 hours.

The preferred concentration of the starting (ω-aminoalkylamino)alkyl alcohol of Formula (I) is in the range from about 0.5 M to about 2.5 M. More dilute solutions can lead to a larger percentage of the free anions, as discussed in Le Noble [Synthesis, 1: p. 1 (1970)]. The preferred amount of halide used in the conversion of the alcohol of Formula (I) to the dihydrohalide of Formula (II) ranges between about a stoichiometric amount to about a several-fold excess, say about a four-fold excess, or more preferably a two-fold excess. The preferred amount of halide used in the conversion of the dihydrohalide of Formula (II) to the halide dihydrohalide of Formula (III) ranges between about a stoichiometric amount and about a two-fold excess.

### B. Amifostine Monohydrate and Trihydrate Preparation and Purification

In a further aspect of the present invention, the (ω-aminoalkylamino)alkyl halides dihydrohalides of Formula (III) can be used to prepare a variety of synthetic products. For example, the compounds of Formula (III) can be used in the manufacture of therapeutically useful compounds, such as the broad class of cytoprotective/radio-protective agents that include amifostine (Ethyol®). These compounds, broadly termed “S-ω-(ω-aminoalkylamino)alkyl dihydrogen phosphorothioates” (Formula IV), can be synthesized according to the process shown in Scheme II.

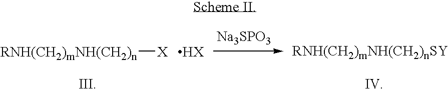

According to this process, compounds of general Formula (III), such as 2-(3-aminopropylamino)ethyl bromide dihyrobromide, can be contacted with sodium thiophosphate for a period of time sufficient to form compounds of Formula (IV) and hydrates thereof.

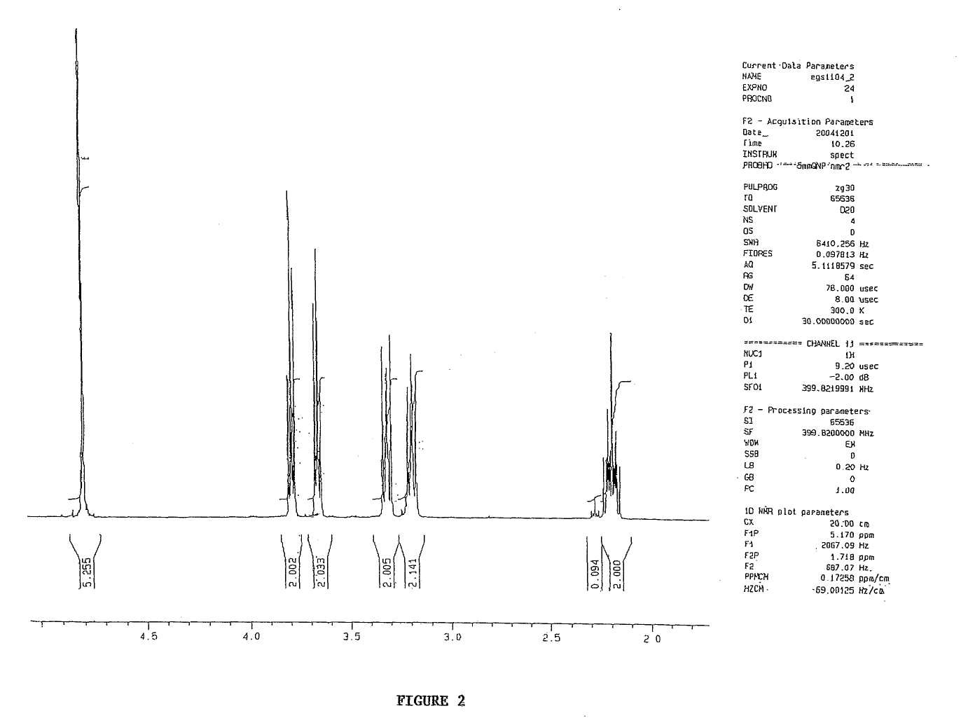

The crude phosphorothioate compounds of Formula IV, such as amifostine, prepared as described above, can be purified to remove color bodies and residual sodium thiophosphate and converted to amifostine monohydrate or trihydrate using the procedure shown in FIG. 2.

Referring generally to FIG. 2, vessel 10 is preferably a jacketed reactor used for dissolving the crude phosphorothioate (i.e., amifostine monohydrate or trihydrate) in water forming an aqueous phosphorothioate solution; however any suitable container may be employed. The aqueous phosphorothioate solution in vessel 10 is pumped through at least two jacketed columns 30 and 40, containing anion-exchange resin and activated carbon, respectively. The columns can be arranged such that the aqueous phosphorothioate solution is pumped through the anion-exchange column first, or the activated carbon column first, with equally acceptable results. Dowex® 1×8-100 (Cl) anion exchange resin and Darco® 20-40 mesh activated carbon granules are suitable materials for columns 30 and 40. Both vessel 10 and columns 30 and 40 are preferably connected to a recirculating chiller (not shown) to allow for temperature control, preferably within the range between about −10° C. and about 30° C.

After the serial treatment in columns 30 and 40, the aqueous phosphorothioate solution is then passed through filter 50, which is preferably a membrane filter having a porosity of about 5 μm or less, to remove any particulate contamination. Following filtration, the aqueous phosphorothioate solution is delivered into vessel 60, which is preferably a stirred reactor. Before receiving the filtered aqueous phosphorothioate solution, vessel 60 is first charged with about 1 vol % to about 60 vol % water in methanol solution, preferably about a 10 vol % water in methanol solution. The filtered aqueous phosphorothioate solution is added to vessel 60 over a period of time from about 0.5 hours to about 6 hours and allowed to mix with the water/methanol solution for a period of time from about 1 hour to about 3 hours. Vessel 60 is then chilled to about 0° C., and its contents are allowed to stand, with optional stirring as necessary, allowing the amifostine monohydrate product to precipitate out of solution. The precipitated monohydrate is collected in filter 70, or alternatively in a centrifuge, or by any other collection means known in the art. Cooling the aqueous phosphorothioate solution in vessel 10 and columns 20 and 30 reduces the rate of hydrolytic decomposition while chilling vessel 60 improves product recovery.

The number of hydrating waters in the crystalline phosphorothioate product may be controlled by adding the filtered aqueous phosphorothioate solution into cold (about 0° C.), aqueous methanol, or by adding seed crystals to vessel 60.

The following examples are included to demonstrate various embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Laboratory Preparation of 2-(3-Amino-propylamino)ethyl Bromide Dihydrobromide

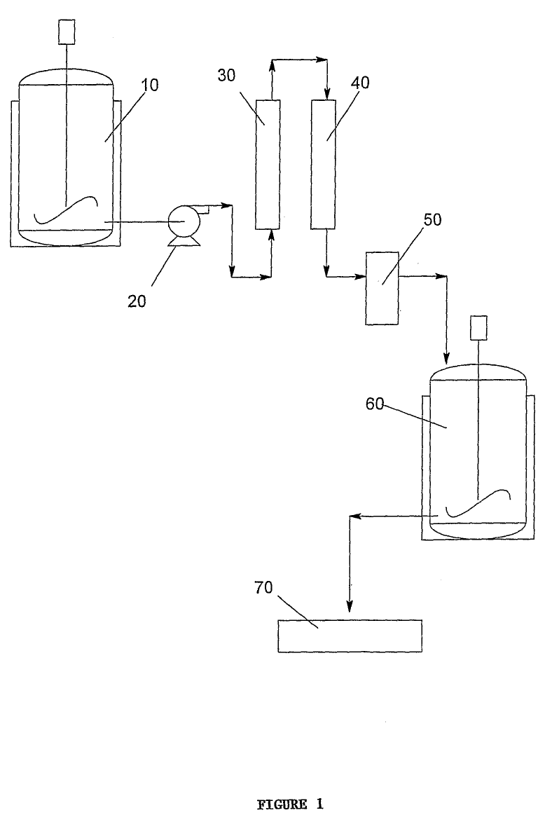

In a nitrogen purged glove box, a 3-Liter, 4-neck round-bottom flask, fitted with a mechanical stirrer, thermocouple, nitrogen inlet adapter and septum, was charged with 2-(3-aminopropylamino)ethanol (144 g; 1.22 mol) and sulfolane (1.00 L; 10.5 mol). This was brought to a fume hood where it was attached to a nitrogen line and the septum was replaced with an adapter holding a ⅛ inch diameter Teflon® tube attached to a HBr lecture bottle. While stirring, the hydrogen bromide (HBr) gas was admitted subsurface at a rate that allowed the temperature to rise to about 130° C. Addition was discontinued after heat evolution ceased and HBr was no longer absorbed; two equivalents had reacted, giving the dihydrobromide salt of the starting alcohol. The adapter on the reaction flask was replaced with a pressure-equalized dropping funnel containing phosphorus tribromide (PBr₃) (132 g, 0.487 mol, 1.2 equiv) which was added over about 10 minutes at a temperature of between about 110° to about 130° C. The solution was then stirred under nitrogen at 120° C. for 20 minutes, after which time the product had crystallized into a thick cake. Additional sulfolane (380 mL) was added, resulting in a slurry which could be stirred at 120° C. After a period of time, the hot slurry was transferred through a ⅜″ polypropylene tube and was dropped into 2 L of acetone, stirring in a 4 L beaker (in three approximately equal portions) to precipitate the product. After each portion, the solid was filtered and rinsed with acetone and the beaker was charged with 2 L of fresh acetone for the next portion. Finally, the round bottom flask was rinsed with acetone and the resulting solid was combined with other portions. The solid was dried by passing nitrogen through the filtration bed overnight, giving 391 g (1.14 mol, 94%) of pale yellow hygroscopic powder. The ¹H NMR showed that it contained residual sulfolane in a 0.023:1 mol ratio (FIG. 1).

Subsequent studies showed that less PBr₃ is needed for this reaction, the 0.33:1 mol ratio required by the stoichiometry is nearly adequate and excess PBr₃ contributes to the formation of colored impurities which are removed from the final product as described below. It was also found that the buildup of product cake after PBr₃ addition can be prevented by increasing the initial sulfolane charge and by maintaining the reactor temperature at about 120° C.

### Example 2

### Synthesis of Sodium Thiophosphate

Sodium thiophosphate and its hydrates were prepared as described in the literature [Inorganic Synthesis, 5: 102 (1957); ibid. 17: 193 (1977)], by reaction of aqueous sodium hydroxide with thiophosphoryl chloride. The only difference was that thiophosphoryl chloride was slowly added to caustic solution at reflux in order to control the exothermic reaction.

### Example 3

### Synthesis of Amifostine Monohydrate

Amifostine was prepared by reaction of equimolar amounts of sodium thiophosphate and 2-(3-aminopropylamino)ethyl bromide dihydrobromide in water as described in U.S. Pat. No. 3,892,824. However, he process and the isolation and purification of the phosphorothioate product were modified. First, a sulfolane solvent was employed, which allowed the intermediate (dihydrohalide) salt to remain substantially in solution and thereby preventing premature precipitation. By keeping the intermediate in solution, conversion of the intermediate to the desired alkyl halide salt was maximized. Second, the HBr/PBr₃/sulfolane reaction produces some colored impurities that must be removed. Finally, the HPLC analytical method required for amifostine, described in the amifostine monograph of the US Pharmacopeia (USP 27, 2004), is very sensitive to traces of thiophosphate salts, due to their high UV extinction coefficients at 220 nm wavelength. In order to meet the purity requirements expressed in Area %, traces of thiophosphate must be minimized. Examples of the purification methods are given below. Also detected by the USP HPLC method is 2-[(3-aminopropyl)amino]ethanethiol, the primary organic hydrolysis product of amifostine, which is referred to below as the thiol.

### Example 4

### Laboratory Purification of Amifostine Monohydrate

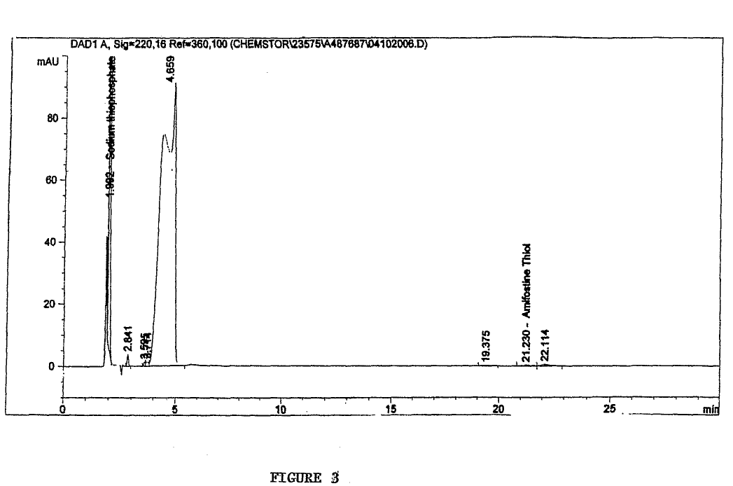

A solution of crude Amifostine was prepared by reacting anhydrous sodium thiophosphate (242 g, 1.34 mol) with 2-(3-aminopropylamino)ethyl bromide dihydrobromide) (470 g, 1.37 mol) in deionized water (1.52 L) at 15° C., the reaction being promoted by DMF (183 g). The crude Amifostine monohydrate was precipitated by slowly adding this solution to a total of 16 L of methanol in three portions, filtered and dried to give 204 g of off-white solid, containing 0.76 water/Amifostine mole ratio by ¹H NMR. HPLC analysis by the USP monograph method (FIG. 3) indicated that the compound was 80.3 A % pure, it contained 18.4 A % thiophosphate and 0.2 A % thiol.

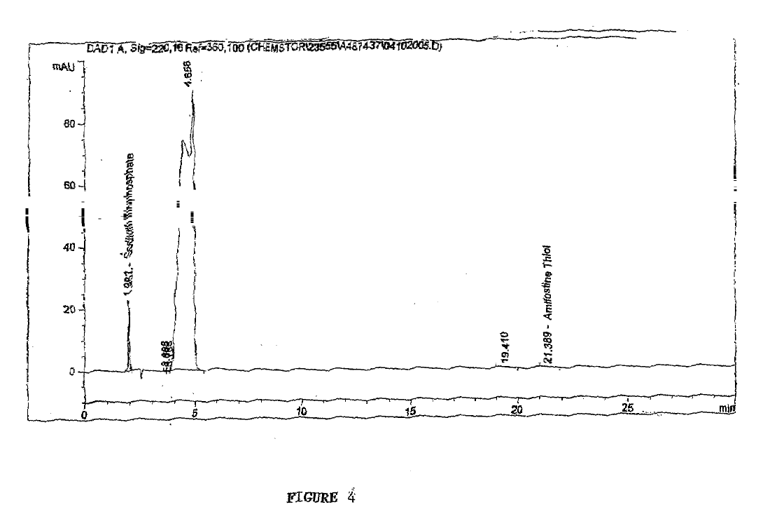

The crude monohydrate was recrystallized to trihydrate by dissolving it in 1.00 L of 10% (v/v) methanol in water at 23° C., adding seed crystals of amifostine trihydrate from a previous batch, and slowly adding methanol (133 mL) to saturate the solution at 25° C. The stirred solution was slowly cooled to 3° C. over 2.5 hours after which the slurry was stirred for 1.5 hours at 0-3° C. The solution was filtered and the solids were rinsed with methanol and dried by passing nitrogen through the filter bed overnight, giving 192 g of crude amifostine trihydrate as slightly brown crystals. This material contained 2.79 water moles/mole of amifostine by ¹H NMR. HPLC analysis by the USP monograph method (FIG. 4) indicated that the compound was 97.5 A % pure, it contained 2.2 A % thiophosphate and 0.1 A % thiol.

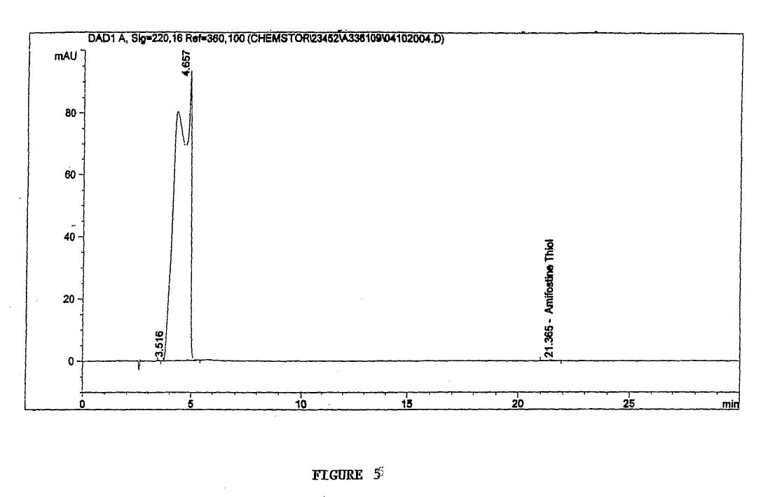

The crude trihydrate was purified and crystallized as monohydrate by dissolving 50 g of the above material in 175 mL of deionized water. The solution was passed through a 1 in. diameter chromatography column and dropped into a stirred beaker of methanol (2.6 L). This column contained 10 g of Darco® activated carbon granules (20-40 mesh) and, in a separate layer, 10 g of Dowex® 1×8-100 (Cl) anion exchange resin. White monohydrate was collected by filtration and dried, 40.2 g. This material contained water to amifostine in a 0.88 mole ratio ¹H NMR. HPLC analysis by the USP method (FIG. 5) indicated that the material was 99.9 A % pure, it contained <0.1 A % thiol and no sodium thiophosphate.

### Example 5

### Synthesis of 2-(3-Aminopropylamino)ethyl bromide dihydrobromide, 1.3 kg Scale

A stirred 20 L glass reactor was charged with sulfolane (14.2 kg) and 2-(3-aminopropylamino)ethanol (1.29 kg, 10.9 mol) at 90° C. The solution was sparged with nitrogen through a Hasteloy C dip-leg, then anhydrous hydrogen bromide (total 1.77 kg, 21.9 mol) was slowly admitted below the liquid surface. The temperature during addition was allowed to rise to 119° C. during addition, the solution was stirred for 15 minutes and was then allowed to stand at 110° C. under nitrogen purge overnight. The solution temperature was raised to 120° C. and, using a Masterflex® pump and ⅛ inch diameterTeflon® tubing, phosphorus tribromide (1.034 kg, 3.82 mol) was added over one hour. The tubing was rinsed into the reactor with more sulfolane (0.60 kg). While stirring rapidly at 120° C., nitrogen was bubbled through the dip-leg for one hour to remove excess HBr.

To a stirred 30 L reactor under nitrogen containing acetone (16.8 kg), one-half of the hot sulfolane solution was transferred using a V₂ inch diameter PTFE tube. The acetone slurry was stirred 15 minutes and then the reactor was drained into a polyethylene bench-top vacuum filtration funnel that was kept under nitrogen using a metal cover. The 30 L reactor was again charged with acetone (16.9 kg), purged with nitrogen, and the remaining hot sulfolane solution was transferred from the 20 L reactor. After stirring, the slurry was discharged and filtered into the same bench-top funnel. The 30 L reactor was charged with more acetone (6.8 kg), purged with nitrogen and heated to 50° C. The hot acetone was carefully drained into the funnel and, under nitrogen, the combined solids were washed and filtered. This hot acetone wash was repeated in order to effectively remove sulfolane. The product was then dried to constant weight under vacuum at about 74° C., giving the dihydrobromide salt (3.58 kg, 10.4 mol, 96% yield).

### Example 6

### Preparation of Sodium Thiophosphate Kilogram Scale

A glass 30 L reactor under nitrogen was charged with deionized water (20 kg) and sodium hydroxide pellets (2.87 kg, 71.8 mol). It was stirred to dissolve and heated to 86° C. Thiophosphoryl chloride (3.59 kg, 11.2 mol) was slowly added using a Masterfex® pump and PTFE tubing over one hour, maintaining a gentle reflux. After stirring for 20 minutes at 95° C., the reactor was cooled to 3° C. over 2 hours and stirred 20 minutes to give a slurry of crystalline sodium thiophosphate dodecahydrate. This was drained into a benchtop funnel, vacuum filtered, washed with 8 then 3 L of cold water and dried under a flow of nitrogen giving 5.37 kg of product containing, by HPLC analysis, 24.5 wt % sodium thiophosphate. The yield on a dry basis was 1.32 kg, 7.33 mol, 65%.

This procedure can be modified by washing the product with methanol to partly or completely dehydrate the solid.

### Example 7

### Preparation of Amifostine, Kilogram Scale

While under nitrogen, a 20 l glass reactor was charged with water (10.3 kg), sodium thiophosphate (1.24 kg on a dry basis, 6.92 mol) and 2-(3-aminopropylamino)ethyl bromide dihydrobromide (2.45 kg. 7.23 mol). The reactor was cooled to 15° C. and DMF (600 g) was pumped in slowly using a Masterflex® pump and PTFE tubing creating an exotherm (to 23° C.) as the reaction commenced. The mixture was stirred 1.5 hours at 15° C.

A 30 L glass reactor was charged with methanol (20 L) which was cooled to 0° C. One third (4.5 L) of the solution in the 20 L reactor was transferred into the 30 L reactor using a ¼ in. PTFE tube, the slurry was drained into a polyethylene bench-top funnel, vacuum filtered and rinsed with methanol (2 L). This procedure was repeated twice, combining the solids in the funnel to give a wetcake of crude monohydrate (3.2 kg) as a light brown powder.

The above wetcake was reintroduced into the 30 L reactor and a solution of 5 wt % methanol in water (10.5 kg) was added. The mixture was heated to 30° C. with stirring to complete dissolution, then seed crystals (about 0.5 g) of Amifostine trihydrate and methanol (0.32 kg) were added to saturate the solution. The solution was cooled with stirring from 30° C. to 0° C. over two hours. The slurry was drained and vacuum filtered using the benchtop funnel, the solid was washed with cold methanol and dried under vacuum at 20° C. to give crude Amifostine trihydrate (1.2 kg) as a light brown crystalline solid.

### Example 8

### Crude Amifostine Trihydrate Purification

A flask was charged with crude amifostine trihydrate (1.734 kg, 6.46 mmol) and deionized water (5.6 L), then briefly warmed (30°-35° C.) with stirring to facilitate dissolution, then cooled to 15° C. A column was packed with activated carbon (55 g) and another column packed with ion exchange resin (100 g). A reactor was charged with methanol (21.17 kg), water (2.30 kg) and was cooled to −2° C. with stirring. It had also been charged with amifostine monohydrate seed crystal (0.5 g). One fourth of the crude amifostine trihydrate solution from the flask (1.7 L) was pumped at 14 mL/min over 2 hours 15 minutes through the carbon and resin columns (15° jacket temperature), was filtered across a membrane and delivered into the reactor containing the methanol/water mixture. Addition was then stopped and the slurry was drained into a table top filter. After vacuum filtration, the wetcake was washed with methanol (1.6 kg) was partially dried by pumping nitrogen through the solid. It was then removed to a vacuum drying oven. The reactor was again loaded with methanol and water as above and the procedure was repeated for a total of four precipitate drops, each time the aqueous solution was pumped over 2-3 hours through the purification beds. After the fourth cycle, the flask, columns and membrane filter were rinsed with water (300 ml) and this was combined in the reactor. The wetcakes were dried under vacuum at 20°-30° C. giving purified amifostine monohydrate (total 1.280 g, 5.51 mol, 85% recovery). This material contained water to amifostine in a 1.02 mole ratio by ¹H NMR. HPLC analysis by the USP method indicated that the material was 99.5 A % pure, it contained 0.2 A % sodium thiophosphate and 0.3 A % thiol was detected.

In some circumstances the wetcakes from the precipitate drops are combined in the same filtration funnel, under a nitrogen atmosphere, before the total wetcake is washed with methanol and dried under vacuum.

After purification with activated carbon and anion exchange resin, the amifostine solution can be converted into a purified trihydrate form. This can be done by recrystallizing the purified monohydrate, as described in Example 9 below, or by directly crystallizing the trihydrate from solution by adding ethanol or methanol nonsolvents, trihydrate seed crystals and then cooling.

### Example 9

### Preparation of Purified Amifostine Trihydrate

Purified amifostine monohydrate (100 g, 0.431 mol) was dissolved in 1000 mL of 5% (v/v) abs. ethanol in D. I. water. The stirred solution was warmed to 31° C., absolute ethanol (360 mL) and seed crystals of the monohydrate were slowly added until saturation was evident.

The slurry was cooled with stirring from 36° to 1° C. over three hours and was then stirred at 1° C. overnight. The slurry was transferred under pressure to a Pyrex® Buchner funnel with a coarse glass frit and suction filtered. The solid was washed with ethanol and dried by passing nitrogen through the wetcake for two hours. It was removed from the funnel giving 105 g of solid product. Quantitative ¹H NMR in D₂O (100% D) showed that it contained 2.79 moles of water per mole of amifostine.

Since the above material contained too little water, it was recrystallized again. A sample (0.6 g) was retained as seed crystals, then the remaining solid was dissolved in 1000 mL of 5% ethanol/water at 33° C. Ethanol (230 mL) and seed crystals were slowly added, and the slurry was cooled to 2° C. over three hours. The solid was filtered as before, but without the ethanol washing step, and was dried with a nitrogen stream for 2 hours. Quantitative ¹H NMR in D₂O (100% D) showed that it contained 2.94 moles of water per mole of amifostine.

While the compositions and methods of this invention have been described in terms of preferred embodiments, it will be apparent to those of skill in the art that variations may be applied to the compositions, methods and/or processes and in the steps or in the sequence of steps of the methods described herein without departing from the concept and scope of the invention. More specifically, it will be apparent that certain agents which are chemically related may be substituted for the agents described herein while the same or similar results would be achieved. All such similar substitutes and modifications apparent to those skilled in the art are deemed to be within the scope and concept of the invention.

What is claimed is:

1. A process for preparing (ω-aminoalkylamino)alkyl halides dihydrohalides, the process comprising the steps of:
contacting, in the presence of a sulfone solvent, an (ω-aminoalkylamino)alkyl alcohol of Formula (I),
RNH(CH₂)ₘNH(CH₂)ₙ—OH (I),
wherein,
R is hydrogen or a substituted or unsubstituted linear, cyclic, or branched alkyl group having 1 to 12 carbon atoms,
m is an integer from 2 to 8, and
n is an integer from 2 to 6,
with a first halogenating agent for a period of time sufficient to provide a dihydrohalide salt of Formula (II),
RNH(CH₂)ₘNH(CH₂)ₙ—OH.2HX (II),
wherein X is a halogen atom; and
contacting, in the presence of a sulfone solvent, the dihydrohalide salt of Formula (II) with a second halogenating agent for a period of time sufficient to provide an (ω-aminoalkylamino)alkyl halide dihydrohalide salt of Formula (III),
RNH(CH₂)ₘNH(CH₂)ₙ—X.2HX (III).

2. The process according to claim 1, wherein the sulfone solvent is sulfolane, dimethylsulfolane, diphenylsulfolane, or mixtures of any two or more of the foregoing.

3. The process according to claim 1, wherein the (ω-aminoalkylamino)alkyl halide dihydrohalide salt of Formula (III) is 2-(3-aminopropylamino)ethyl bromide dihyrobromide.

4. The process according to claim 1, wherein the contacting of the (ω-aminoalkylamino)alkyl alcohol of Formula (I) and the contacting of the dihydrohalide salt of Formula (II) are carried out at a temperature in a range from about 100° C. to about 150° C.

5. The process according to claim 1, wherein the first halogenating agent is hydrogen bromide.

6. The process according to claim 1, wherein the second halogenating agent is phosphorus tribromide or phosphorus pentabromide.

7. The process of preparing S-ω-(ω-aminoalkylamino)alkyl dihydrogen phosphorothioates comprising the steps of:
contacting, in a sulfone solvent, an (ω-aminoalkylamino)alkyl alcohol of Formula (I),
RNH(CH₂)ₘNH(CH₂)ₙ—OH (I),
wherein,
R is hydrogen or a linear, cyclic, or branched alkyl group having 1 to 12 carbon atoms, which can be substituted or unsubstituted,
m is an integer from 2 to 8, and
n is an integer from 2 to 6,
with a first brominating agent for a period of time sufficient to provide a dihydrobromide salt of Formula (II),
RNH(CH₂)ₘNH(CH₂)ₙ—OH.2HBr (II);
contacting, in the presence of a sulfone solvent, the dihydrobromide salt of Formula (II) with a second brominating agent for a period of time sufficient to provide an (ω-aminoalkylamino)alkyl bromide dihydrobromide salt of Formula (III),
RNH(CH₂)ₘNH(CH₂)ₙ—X.2HX (III),
wherein X is a bromine atom;
isolating the dihydrobromide salt of Formula (III); and
contacting the dihydrobromide salt of Formula (III) with sodium thiophosphate for a period of time sufficient to form compounds of Formula (IV),
RNH(CH₂)ₘNH(CH₂)ₙSY (IV),
and hydrates thereof, wherein R, m, and n or as previously defined and Y is PO₃H₂, PO₃HM, or PO₃M₂, wherein M is an alkali metal selected from sodium, potassium, and lithium.

8. The process according to claim 7, wherein the S-ω-(ω-aminoalkylamino)alkyl dihydrogen phosphorothioate is amifostine.

9. A process for the preparation of an aqueous purified amifostine solution from crude amifostine, the process comprising:
preparing an aqueous crude amifostine solution by mixing crude amifostine in water, wherein the crude amifostine was prepared by brominating an amino alcohol at least in part with a phosphorus bromide in a sulfone solvent; and
contacting the aqueous crude amifostine solution with at least one ion exchange column and at least one activated carbon column thereby forming an aqueous purified amifostine solution.

10. The process of claim 9, further comprising the step of precipitating amifostine monohydrate from the aqueous purified amifostine solution by contacting the aqueous purified amifostine solution with a water-methanol mixture over a period of time from about 0.5 h to about 9 h, the water-methanol mixture comprising about 1% to about 60% volumetric excess of methanol relative to the water.

11. The process of claim 9, further comprising filtering, washing, and drying the amifostine monohydrate.

12. The process of claim 10, wherein the water-methanol mixture comprises a 10% to a 40% volumetric excess of methanol relative to the water.

13. A process for the preparation of (ω-aminoalkylamino)alkyl bromides, the process comprising:
a. contacting an (ω-aminoalkylamino)alkyl alcohol with hydrogen bromide in the presence of a sulfone solvent at a temperature from about 100° C. to about 150° C. for a period of time sufficient to provide a dihydrobromide salt of the (ω-aminoalkylamino)alkyl alcohol;
b. contacting the dihydrobromide salt of the (ω-aminoalkylamino)alkyl alcohol in the sulfone solvent with a brominating agent to form an (ω-aminoalkylamino)alkyl bromide dihydrobromide salt; and
c. precipitating the (ω-aminoalkylamino)alkyl bromide dihydrobromide.

14. The process of claim 13, wherein the sulfone solvent is sulfolane.

15. The process of claim 13, wherein the reaction is carried out at a temperature of about 100° C. to about 150° C.

16. The process of claim 13, wherein the brominating agent is phosphorus tribromide or phosphorus pentabromide.
